(19) United States
(12) Patent Application Publication
(10) Pub. No.: US 2024/0242819 A1
(43) Pub. Date: Jul. 18, 2024

(54) METHOD FOR AI APPLICATIONS IN MRI SIMULATION

(71) Applicant: Corsmed AB, Stockholm (SE)

(72) Inventors: Christos XANTHIS, Athens (GR); Anthony ALETRAS, Thessaloniki (GR)

(21) Appl. No.: 18/425,545

(22) Filed: Jan. 29, 2024

Related U.S. Application Data
(63) Continuation-in-part of application No. 16/927,930, filed on Jul. 13, 2020.

(51) Int. Cl. G16H 30/40; G06F 30/20

(52) U.S. Cl. CPC G16H 30/40; G06F 30/20

(57) ABSTRACT

A method for generating training datasets for AI applications includes providing an MRI simulator and input thereto. The input is a pulse sequence and an anatomical model. The method includes selecting the position and/or orientation of a plane/volume of interest of the anatomical model, and executing the MRI simulator, producing a simulated MR image. The method includes obtaining produced synthesized MR images and/or label maps for each synthesized MR image, obtaining a training dataset based on all obtained produced MR images and/or label maps possible to produce for each MR image, and building a computer model for synthesized MR images and/or label maps using an SBR framework. The procedure may be repeated with different pulse sequences and/or the same pulse sequence, by amending the position and/or orientation of a plane/volume of interest of the anatomical model, and/or by amending properties of the anatomical model, and/or with another anatomical model.

### CROSS-REFERENCE TO RELATED APPLICATION

This application is a continuation-in-part of co-pending U.S. patent application Ser. No. 16/927,930, filed Jul. 13, 2020, which is incorporated herein by reference in its entirety.

### FIELD OF THE INVENTION

The present invention relates to a method for generation of training datasets for artificial intelligence applications in magnetic resonance imaging.

### SUMMARY OF THE INVENTION

The present invention is directed to a method for generation of training datasets for artificial intelligence (AI) applications in MRI (Magnetic Resonance Imaging), said method comprising
providing an MRI simulator;
providing input to the MRI simulator, said input being in the form of a pulse sequence and a computer-based anatomical model;
selecting the position and/or orientation of a plane/volume of interest of the anatomical model;
executing the MRI simulator and thus producing a simulated artificial MR image;
obtaining produced synthesized MR images and/or label maps for each synthesized MR image;
obtaining a training dataset based on all obtained produced MR images and/or label maps possible to produce for each MR image,
wherein the method involves repeating the same procedure with different pulse sequences and/or the same pulse sequence, by amending the position and/or orientation of a plane/volume of interest of the anatomical model, and/or by amending properties of the anatomical model, and/or with another anatomical model, said method also comprising
building a computer model for synthesized MR images and/or label maps using a simulation-based reconstruction (SBR) framework.

As should be clear from above, the present invention is directed to AI applications for MRI. In particular, the method according to the present invention relates to using simulated MR images, i.e. artificial images, for training AI, i.e. obtaining a training dataset.

Furthermore, the method according to the present invention involves a repetition step in which something is changed in relation to the pulse sequence and/or the anatomical model and/or the characteristics of the MR simulation used in the first simulation. Either a different pulse sequence is used, or the characteristics of the same pulse sequence are altered. Another option is of course to both use a different pulse sequence and also amend the characteristics of this pulse sequence when comparing with the characteristics or design of the first pulse sequence used. It should be noted that the method according to the present invention may involve repeating the same procedure with different computer-based anatomical models or the same anatomical model at a different state (for example with the addition of pathology or at a different cardiac or respiratory cycle).

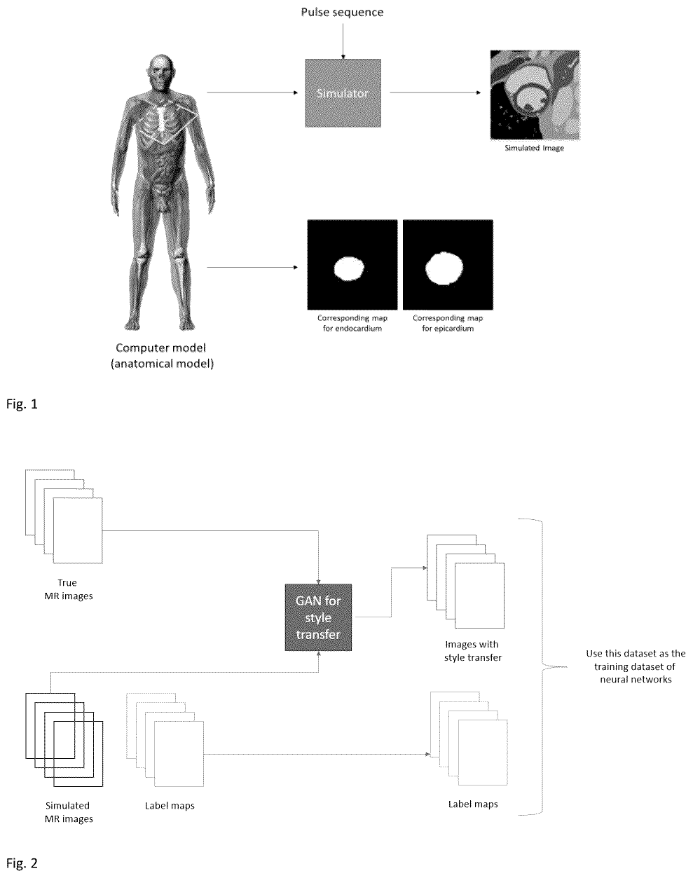

Moreover, in relation to the possible step of producing a label map, the expression “label map” may also be stated as “annotation map”. An example of a label map is shown in FIG. 1. In this example, the label map is of the same size as the simulated image and tells you if a pixel of the simulated image belongs to a tissue type (or tissue area) or not. It should be noted that it may also be of a different type, such as a label that says that in this image there is a tissue type (for example a tumor) or not. Depending on the application, it may have a different form.

### SPECIFIC EMBODIMENTS OF THE INVENTION

Below some specific embodiments of the present invention are disclosed and explained further.

According to one specific embodiment, using the simulation-based reconstruction (SBR) framework comprises utilizing a pulse sequence or a set of pulse sequences and large-scale nonlinear optimization to reconstruct the quantitative parameter maps of the underlying tissue properties of the anatomical model. Large-scale nonlinear optimization refers to the process of finding the optimal solution for a problem where the objective function exhibits nonlinearity, and the problem is characterized by a large number of variables and potentially complex relationships between them. According to the present invention, the method is directed to finding the best solution (e.g. tissue properties) that minimizes the objective function (in SBR, this is the difference between the true MR signal and the simulated MR signal for the same experiment). In line with this, according to one embodiment, the step of building a computer model for synthesized MR images and/or label maps using a simulation-based reconstruction (SBR) framework comprises determining the difference between the true MR signal and the simulated MR signal for the same experiment.

According to yet another embodiment, wherein the large-scale nonlinear optimization is performed, the spin dynamics are simulated according to the physical models that govern the evolution of the acquired MR signal (forward model). According to one specific embodiment of the present invention, the step of obtaining produced synthesized MR images and/or label maps for each synthesized MR image is both performed in the MRI simulator. In relation to this it should be noted that according to the present invention, the “MRI simulator” is a software. This further implies that the step of producing a label map for each MR image may be produced by this software, but this does not imply that MR simulation is performed in this step.

Moreover, according to another embodiment of the present invention, the method comprises a step for obtaining produced synthesized MR images and a step for obtaining label maps for each synthesized MR image, preferably wherein both steps are performed in connection to each other, more preferably simultaneously or alternately. Suitably, once the position of the slice-of-interest in 3D space is defined, then the production of images and label maps can run in parallel.

According to one embodiment, the method comprises repeating the same procedure with different pulse sequences and/or the same pulse sequence. According to one specific embodiment, the method according to the present invention involves repetition by using a different pulse sequence or amending the characteristics of the same pulse sequence, or both. Furthermore, according to one specific embodiment, the method comprises amending the characteristics of the pulse sequence and/or amending the characteristics of the MR simulation when executing the MRI simulator. Examples of parameters included in the characteristics of the pulse sequence are repetition time (TR), matrix size, and receiver BW (bandwidth).

Moreover, according to one specific embodiment, the properties of the anatomical model are also amended. According to one specific embodiment, the method comprises amending the properties of the anatomical model, and wherein these properties are at least T1 and/or T2 properties of the tissues (the underlying pixels). Moreover, and as said, according to yet another embodiment, the position and/or orientation of a plane/volume of interest of the anatomical model is also amended. It should again be said that the method according to the present invention covers the option of using a different anatomical model or the same anatomical model at a different state (for example with the addition of pathology or at a different cardiac or respiratory cycle).

According to yet another embodiment, the method involves amending the characteristics of the MR simulation/experiment when executing the MRI simulator. Non-limiting examples of parameters are the B0 inhomogeneity, noise, artefacts, MR hardware imperfections, etc.

According to yet another embodiment, the method involves repeating the same procedure with different pulse sequences and/or the same pulse sequence, amending the characteristics of the anatomical model, and amending the position and/or orientation of a plane/volume of interest of the anatomical model.

It should again be said that according to another specific embodiment of the present invention, the method comprises using another anatomical model and repeating the same procedure with said another anatomical model.

As hinted above, the MRI simulator according to the present invention suitably is a software. According to one specific embodiment, the MRI simulator is web-based and cloud-based. This has advantages both for the user as such and in relation to data handling, transfer and storage.

Furthermore, according to yet another implementation embodiment of the present invention, the method involves simulation of a magnetic resonance (MR) scanner in the MRI simulator, said method comprising
input of data parameters into a web interface of the MRI simulator;
connection of the web interface with a cloud-based simulator engine of the MRI simulator for transfer of data parameters to the cloud-based simulator engine;
recalculation of the data parameters for the provision of one or more simulated MR signals, said recalculation being performed in the cloud;
reconstruction of an MR image based on said one or more simulated MR signals, said reconstruction of an MR image being performed in the cloud; and
sending the MR image to the web interface.

In relation to the web interface of the MRI simulator it should be noted that a corresponding web-service may be used instead of a specific web interface. Furthermore, in relation to the last step of sending the MR image to the web interface it should be noted that this step may also be performed instead by calling a web-service.

Furthermore, according to one specific embodiment, the cloud-based simulator engine performs the recalculation and sends recalculated data to one or more GPUs (graphics processing units) of the MRI simulator, which GPUs sends back said one or more simulated MR signals. Furthermore, according to yet another embodiment, the step of reconstruction of an MR image is performed by one or more CPUs (central processing units) and/or one or more GPUs (graphics processing units) of the MRI simulator in the cloud.

### DESCRIPTION OF THE DRAWINGS AND FURTHER EXPLANATION

In FIG. 1 there is shown one schematic implementation for the method according to the present invention. According to a simplified version, the simulator accepts two first inputs: the pulse sequence and the computer (anatomical model). For every execution of the simulator, a simulated (artificial) MR image is produced. Other possible input parameters are configuration parameters e.g. parameters that define how the MR scanner works, such as type of coil, position of coil, hardware imperfection, etc. The suggested methodology provides a flexible solution that allows the generation of training datasets for multiple variations of both the imaging protocol and the computer model, which is not easy today with a real MRI experiment configuration (MRI system, patient recruitment, etc.). In addition, the digitized and highly-customizable nature of the anatomical model allows for the concurrent production of well-annotated data in the form of tissue masks. The annotation of the artificial data is always objective and depicts the real tissue characteristics without being affected by various factors that may deteriorate the quality of the medical image, such as noise, limited spatial and temporal resolution due to the pulse sequence design, etc. Variants where noise and the presence of other artefacts that can make the data look close to real can easily be accommodated.

### As explained above, the method according to the present invention involves repeating the same process where conditions are altered. The repetition is performed for different:

Pulse sequences or configuration of the same pulse sequence
Characteristics of the anatomical model
Position and/or orientation of the plane/volume of interest
Characteristics of the MR experiment such as the B0 inhomogeneity, noise, artefacts, etc.

Again, and as mentioned, the present invention covers the case of using a different anatomical model or the same anatomical model at a different state.

A set of hundreds of artificial MR images and the corresponding map(s) are produced, and they are used as a training dataset for training a neural network. The neural network is then tested on true MR images (images from patients and volunteers).

In FIG. 2 there is shown a schematic view of the concept according to the present invention. The computer-based anatomical model has been developed based on a simulation-based reconstruction framework. The simulation-based reconstruction framework utilizes properly designed RF excitation and gradient acquisition schemes (pulse sequence) and a large-scale nonlinear optimization problem to accurately reconstruct the quantitative parameter maps of the underlying tissue properties. The pulse sequences are based on designs that encode multiple quantitative parameters into the measured signal that has been acquired from a real MR scanner simultaneously. The large-scale nonlinear optimization solver then performs quantitative reconstructions through an iterative optimization process (inverse problem) where the spin dynamics are simulated according to the physical models that govern the evolution of the acquired MR signal (forward model). The utilization of the simulation-based reconstruction framework allows for the reconstruction of accurate quantitative maps within shorter acquisition times.

On the SBR side, the method involves to reverse-engineer the scanner, but with the objective of extracting the fundamental properties of the body from the acquired signal. The incorporation of accurate physical models that govern the evolution of the acquired MR signal into this process allows for the reconstruction of the quantitative parameter maps of the underlying tissue properties.

To involve SBR technology as suggested according to the present invention, it is possible to build accurate and realistic computer models of subjects quickly, i.e. when building computer models for synthesizing MR images.

1. A method for generation of training datasets for artificial intelligence (AI) applications in MRI (Magnetic Resonance Imaging), said method comprising
providing an MRI simulator;
providing input to the MRI simulator, said input being in the form of a pulse sequence and a computer-based anatomical model;
selecting the position and/or orientation of a plane/volume of interest of the anatomical model;
executing the MRI simulator and thus producing a simulated artificial MR image;
obtaining produced synthesized MR images and/or label maps for each synthesized MR image;
obtaining a training dataset based on all obtained produced MR images and/or label maps possible to produce for each MR image,
wherein the method involves repeating the same procedure with different pulse sequences and/or the same pulse sequence, by amending the position and/or orientation of a plane/volume of interest of the anatomical model, and/or by amending properties of the anatomical model, and/or with another anatomical model, said method also comprising
building a computer model for synthesized MR images and/or label maps using a simulation-based reconstruction (SBR) framework.

2. The method according to claim 1, wherein using the simulation-based reconstruction (SBR) framework comprises utilizing a pulse sequence or a set of pulse sequences and large-scale nonlinear optimization to reconstruct the quantitative parameter maps of the underlying tissue properties of the anatomical model.

3. The method according to claim 2, wherein the large-scale nonlinear optimization is performed with iterative optimization and based on inverse problem, preferably wherein the spin dynamics are simulated according to the physical models that govern the evolution of the acquired MR signal (forward model).

4. The method according to claim 1, wherein the step of building a computer model for synthesized MR images and/or label maps using a simulation-based reconstruction (SBR) framework comprises determining the difference between the true MR signal and the simulated MR signal for the same experiment.

5. The method according to claim 1, wherein the step of obtaining produced synthesized MR images and/or label maps for each synthesized MR image is performed in the MRI simulator.

6. The method according to claim 1, wherein the method comprises a step for obtaining produced synthesized MR images and a step for obtaining label maps for each synthesized MR image, preferably wherein both steps are performed in connection to each other, more preferably simultaneously or alternately.

7 The method according to claim 1, wherein the method comprises
repeating the same procedure with different pulse sequences and/or the same pulse sequence.

8. The method according to claim 1, wherein the method comprises
amending the characteristics of the pulse sequence and/or amending the characteristics of the MR simulation when executing the MRI simulator.

9. The method according to claim 1, wherein the method comprises
amending the properties of the anatomical model, and wherein these properties are at least T1 and/or T2 properties of the tissues.

10. The method according to claim 1, wherein the method involves repeating the same procedure with different pulse sequences and/or the same pulse sequence, amending the characteristics of the anatomical model, and amending the position and/or orientation of a plane/volume of interest of the anatomical model.

11. The method according to claim 1, wherein the method comprises using another anatomical model and repeating the same procedure with said another anatomical model.

12. The method according to claim 1, wherein the MRI simulator is web-based and cloud-based.

13. The method according to claim 12, wherein the method involves simulation of a magnetic resonance (MR) scanner in the MRI simulator, said method comprising
input of data parameters into a web interface of the MRI simulator;
connection of the web interface with a cloud-based simulator engine of the MRI simulator for transfer of data parameters to the cloud-based simulator engine;
recalculation of the data parameters for the provision of one or more simulated MR signals, said recalculation being performed in the cloud;
reconstruction of an MR image based on said one or more simulated MR signals, said reconstruction of an MR image being performed in the cloud; and
sending the MR image to the web interface.

14. The method according to claim 13, wherein the cloud-based simulator engine performs the recalculation and sends recalculated data to one or more GPUs (graphics processing units) of the MRI simulator, which GPUs sends back said one or more simulated MR signals.

15. The method according to claim 13, wherein the step of reconstruction of an MR image is performed by one or more CPUs (central processing units) and/or one or more GPUs (graphics processing units) of the MRI simulator in the cloud.
